(19) United States
(12) Patent Application Publication
(10) Pub. No.: US 2019/0380731 A1
(43) Pub. Date: Dec. 19, 2019

(54) Method and Article for Removing Splinters with a Disposable Lancet

(71) Applicant: DWIGHT DARWIN ALEXANDER, LEWISBURG, PA (US)

(72) Inventor: DWIGHT DARWIN ALEXANDER, LEWISBURG, PA (US)

(21) Appl. No.: 16/010,433

(22) Filed: Jun. 16, 2018

(51) Int. Cl. A61B 17/3205; A61B 17/30

(52) U.S. Cl. CPC A61B 17/3205; A61B 2017/0023; A61B 17/30

(57) ABSTRACT

A method and article for removing splinters is provided. First a generally flat lancet is removed from its packaging. The disposable lancet is then used to pierce the patients skin and loosening the skin in the area adjacent to the splinter before tearing the skins with a sharp point of the lancet. Then the lancet is bent at bend line located at center between both end points till the points of each end are parallel and aligned so their able grasp objects between the points. The lancet should look and function more so like tweezers after the previous step. The foreign object can now be removed, thereafter the lancet, packaging and splinter is now disposed of.

### CROSS REFERENCE TO RELATED APPLICATIONS

Referring to U.S. Pat. No. 5,334,195A, 1994 Aug. 2, Petter Gollobin, Method and article for removing slinters with a disposable lancet. I believe my method and article is patentable because its different shape improves the purpose and operativeness of these prior disposable pre sterilized lancets method.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The invention relates to an improved method and article for removing foreign objects commonly called “splinters”. More particularly, the invention relates to such a method utilizing a pre-sterilized, individually wrapped, disposable lancet having flat points.

The method for removing splinters according to prior art is well known. It is very common for a person who wishes to remove a splinter or other small foreign objects from the skin of themselves, other persons, or an animal to use a pre-sterilized lancet which they attempt to use the pre-sterilized lancets flat point.

There is a basic problem in this method of removing foreign objects. The lancet has a handle portion at one end and a flat point at its other end. The splinter is then removed from the patient's skin by effective manipulation of the flat point of the lancet relative to the splinter, which is good for piercing and loosening the skin around the foreign object so that it can be removed, but not very good for pulling out foreign objects.

Referring now in detail to the drawings and, in particular to FIGS. 1-5, there is illustrated a disposable lancet 10, according to the prior invention. This type of lancet is preferred for use in the method embodying the prior invention, for removing foreign objects from skin. The lancet is made from, for example, corrosion resistant stainless steel. There are many types of lancets which can be employed in connection with the invention. The lancet has a handle area 12 with raised embossed knurls 13 and a point 14. The shape of the point is made up of intersecting flat planes from either a stamping process or grinding process. Up until now, this type of lancet was used to puncture skin in order to draw blood for blood tests or to scoop out foreign objects. A pre-sterilized lancet would be removed from its individual packet and used to prick the finger of the person whose blood needs testing or used to pierce, tear and scoop remove splinters from the skin. The lancet would then be discarded.

It was surprisingly found that although this type of lancet has been used for years to draw blood and splinter removal, it has never been utilized for splinter or other foreign object removal, while its flat surface and blade or opposing blades are ideal for this purpose. The disposable lancet 10 ideally is packaged in an individual pre-sterilized packet. The packets could be sold, for example, in a small container with ten or twenty disposable lancets. The lancet is removed from the packet and held by handle area 12 which may be provided with raised embossed knurls 13 of any desired design which will aid in gripping and also add to the rigidity of the lancet.

An effective lancet according to the invention will have the following general dimensions: the flat point can range in length from 2/64″to 5/16″; the overall length can range from 0.375″to 2.50″; the width can range from ⅛″ to ⅝″; and the thickness of the lancet, excluding any raised gripping portions, can range from 0.003″ to 0.015″.

The following dimensions are set forth as the preferred dimensions for one type of lancet (as shown in the drawings), but they are not intended to limit the invention. The lancet ideally has a length of 1 21/32″± 1/32″, inclusive of the point. The width is approximately 15/64″ and the height 0.007″ non-inclusive of the raised knurls. The total height including the raised knurls would be in the range of 0.025″-0.032″.

A single row of knurls lies in the center of the handle area and is approximately 0.07″ wide and extends 5/16″ along the length. Each knurl has an area of 1/16″ by 1/16″.

The length of the point is 7/64″± 1/64″. The point, as stated above, is in the shape of intersecting flat planes. The planes should be offset with regard to each other 25°±3°. In the event that a long point is desired, its point length would be 5/32″± 1/64″.

Returning now to a description of the method, the sterilized lancet would be removed from its package and the user, grasping the handle area 12 of lancet 10 in a somewhat pen-like grip, would manipulate the lancet so that the point 14 is brought toward the skin area 15 containing the foreign object, as shown in FIG. 5, in the direction of arrow A. The user then applies sufficient pressure on the handle (in the direction of arrow A) such that point 14 then pierces the skin just under the foreign object 16. Since point 14 is a flat surface, when the point is manipulated in an upward direction away from the skin (shown by the direction of arrow D) it can actually pull out the foreign object. A conical needle or pin cannot easily pull an object out because it has a round polished and tapering surface area and the object will tend to slide off the point.

Lancet 10 can also be used to loosen the skin around the splinter or foreign object. By piercing the skin adjacent to the foreign object and then lifting the lancet 10 in a direction perpendicular to the entry direction, the flat blades 17 and 18 can easily tear the patient's skin in directions B and C in FIG. 5. This can open the area adjacent to the foreign object or directly above it and thereby loosen it. Such a tearing action, by a needle or pin perpendicular to the direction of entrance of the needle or pin is not as effective due to its shape. Skin and foreign objects will tend to easily slide off the tapering polished conical point of a needle or pin instead of tearing the skin, as with a flat point.

Once the skin surrounding the foreign object has been loosened up, lancet 10 can be utilized to lift or scoop the foreign object out by lifting away from the skin.

In this regard, the lancet can also be manipulated to pierce the skin, adjacent and parallel to the splinter, in the direction of arrow A. The rear end of handle area 12 can then be pivoted downwardly away from the splinter (as shown by direction D), causing point 14 to move into or under the splinter. Removal of the lancet at this point makes it highly likely that the splinter can be dislodged.

### BRIEF SUMMARY OF THE INVENTION

It is therefore an object of the present invention to provide a method where a splinter or foreign object may be easily removed with a pre-sterilized instrument. It is a further object that such method provides an individually wrapped, disposable sterilized lancet which is easily manufactured and relatively inexpensive.

These and other related objects are achieved according to the invention by a method for removing a splinter from a patient's skin. The method includes the steps of piercing and loosen the patient's skin in an area adjacent to the splinter (i.e., underneath or above the splinter or foreign matter) with a hand-held, disposable, elongated lancet. The lancet has a handle portion between ends and a flat point at both ends. Before the splinter is removed its body is bent at the kurf cut line by applying pressure to both handles. In its alternative form, the handle is now tapered “V-shaped”. The now reliant based handles include the flat points on both ends. The splinter can be grasped between the flat points then removed from the patient's skin by effectively manipulating lancet relative to the splinter.

Preferably, the lancet is a metal lancet, made from corrosion resistant stainless steel, and its flat points has one or two sharpened edges. The lancet is most desirably provided with a set of raised knurls along the handle portion to aid in manipulating the device. The thickness of the lancet, excluding the raised knurls.

In a particularly preferred embodiment, the method includes the step of tearing the patient's skin in an area adjacent to (i.e., above or underneath) the splinter. Such a tearing is achieved by piercing the skin with a point of the lancet adjacent to or above or below the splinter and then pulling the points away from the skin in a direction perpendicular to the entry direction. Because of the flat shape of the points, either the skin tears easily to loosen the splinter before the lancet is folded between the two points by applying pressure to the reliant based handles and utilized like tweezers to grasp and pull or lift the splinter out directly.

Other objects and features of the present invention will become apparent from the following detailed description, considered in connection with the accompanying drawings which disclose the embodiments of the invention. It is to be understood, however, that the drawings are designed for the purpose of illustration only, and not as a definition of the limits of the invention.

### BRIEF DESCRIPTION OF THE SEVERAL VIEWS OF THE DRAWING

In the drawings, wherein similar reference characters denote similar elements throughout the several views:

FIG. 1 is a prior art perspective view of the disposable lancet according to the present invention;

FIG. 2 is a prior art top elevation view of the disposable lancet according to the present invention;

FIG. 3 is a prior art left side view of the disposable lancet according to the present invention;

FIG. 4 is a prior art bottom side elevational view of the disposable lancet according to the present invention;

FIG. 5 is a prior art perspective view of the disposable lancet in use according to the present invention;

FIG. 6 is a top elevation view of the new disposable lancet in a unfolded position for use;

FIG. 7 is a side elevation view of the new disposable lancet in a unfolded position for use;

FIG. 8 is a perspective elevation view of the new disposable lancet in a unfolded position for use;

FIG. 9 is a front elevation view of the new disposable lancet in a unfolded position for use;

FIG. 10 is a perspective view of the new disposable lancet in a unfolded position in use in present method;

FIG. 11 is a side view of the same disposable lancet in a semifolded position;

FIG. 12 is a side elevation view of the new disposable lancet in a folded position for use;

FIG. 13 is a top elevation view of the new disposable lancet in a folded position for use;

FIG. 14 is a perspective view of the new disposable lancet in a folded position for use;

FIG. 15 is a back elevation view of the new disposable lancet in a folded position for use;

FIG. 16 is a front elevation view of the new disposable lancet in a folded position for use; and

FIG. 17 is a perspective view of the new disposable lancet in a folded position for use in present method;

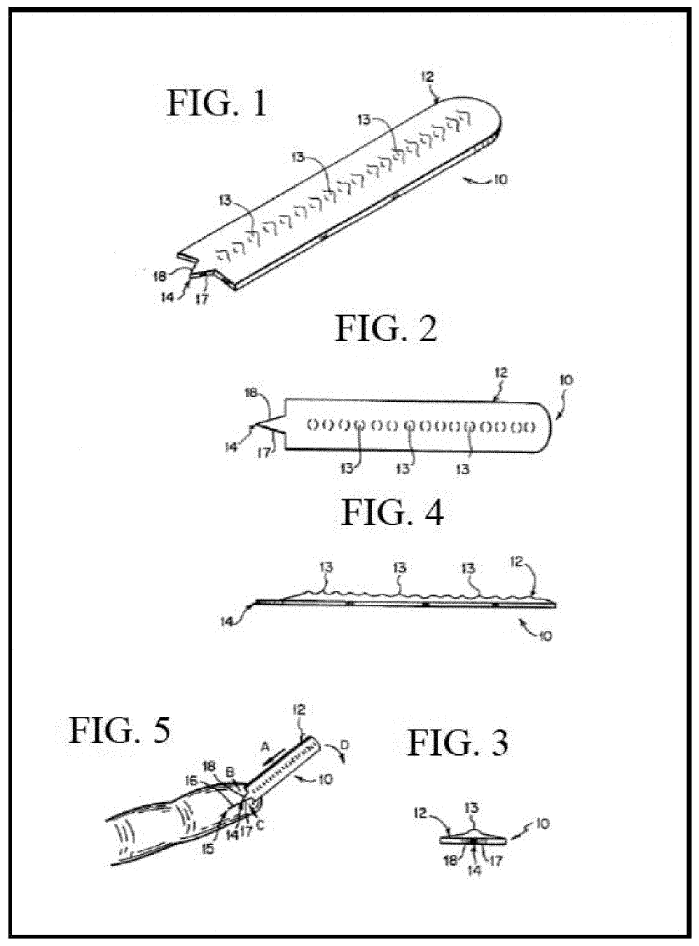

### DETAILED DESCRIPTION OF THE INVENTION

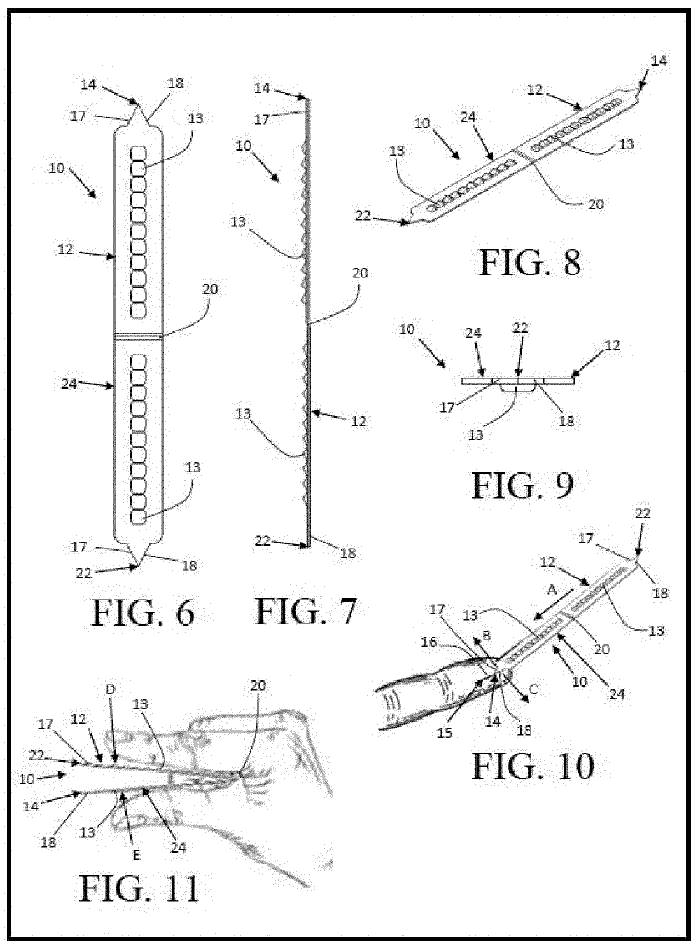

Referring now in detail to the drawings and, in particular to FIGS. 6-17, there is illustrated a disposable lancet 10, according to the present invention. This type of lancet is preferred for use in the method embodying the present invention, for removing foreign objects from skin. The lancet is made from, for example, corrosion resistant stainless steel. The lancet has a handle area 12 and 24 with raised embossed knurls 13 with kurf cut line 20 and points 14 and 22. A pre-sterilized lancet would be removed from its individual packet and used to prick the finger of the person whose blood needs testing or to remove splinters. The lancet would then be discarded.

It was surprisingly found that although this type of lancet has been used for years to draw blood and remove splinters, it has never been designed to be utilized for splinter or other foreign object removal, while its flat surface and blade or opposing blades are ideal for this purpose. The disposable lancet 10 ideally is packaged in an individual pre-sterilized packet. The packets could be sold, for example, in a small container with ten or twenty disposable lancets. The lancet is removed from the packet and held by handle areas 12 and 24 which may be provided with raised embossed knurls 13 of any desired design which will aid in gripping and also add to the rigidity of the lancet.

An effective lancet according to the invention will have the following general dimensions: the flat point can range in length from 2/64″ to 5/16″; the overall length can range from 0.75″to 5″; the width can range from ⅛″ to ⅝″; and the thickness of the lancet, excluding any raised gripping portions, can range from 0.003″ to 0.015″.

The following dimensions are set forth as the preferred dimensions for one type of lancet (as shown in the drawings), but they are not intended to limit the invention. The lancet ideally has a length of 3 5/16″± 1/16″, inclusive of the point. The width is approximately 15/64″ and the height 0.007″ non-inclusive of the raised knurls. The total height including the raised knurls would be in the range of 0.025″-0.032″.

A single row of knurls lies in the center of the handle area and is approximately 0.07″ wide and extends 5/16″ along both lengths except where the kurf cut line is. Each knurl has an area of 1/16″ by 1/16″.

The length of the point is 7/64″± 1/64″. The point, as stated above, is in the shape of intersecting flat planes. The planes should be offset with regard to each other 25°±−3°. In the event that a long point is desired, its point length would be 5/32″± 1/64″.

Returning now to a description of the method, the sterilized lancet would be removed from its package and the user, grasping the handle areas 12 an 24 of lancet 10 can be used to loosen the skin around the splinter or foreign object 16. By piercing the skin adjacent to the foreign object and then lifting the lancet 10 in a direction perpendicular to the entry direction, the flat blades 17 and 18 can easily tear the patient's skin in directions B and C in FIG. 10. This can open the area adjacent to the foreign object or directly above it and thereby loosen it. Such a tearing action, by a needle or pin perpendicular to the direction of entrance of the needle or pin is not as effective due to its shape. Skin and foreign objects will tend to easily slide off the tapering polished conical point of a needle or pin instead of tearing the skin, as with a flat point.

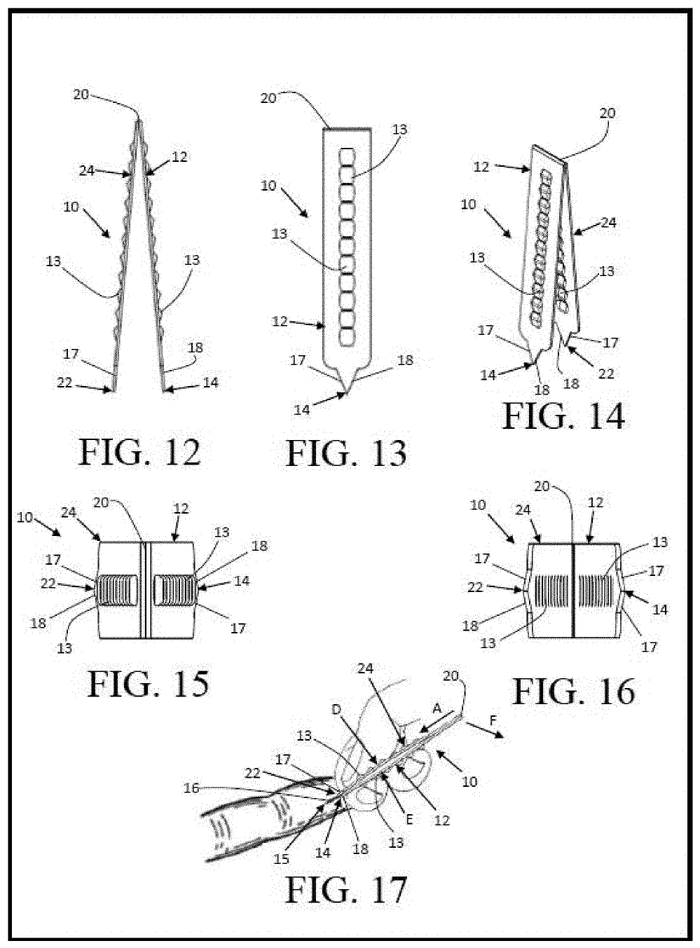

Once the skin surrounding the foreign object has been loosened up, FIG. 11. Shows the lancet being folded by the user applying sufficient pressure on the handles (in the direction of arrow D and E). FIG. 12-17 lancet 10 is showing a folded lancet at the kurf cut line 20 till points 22 and 14 are parallel.

The folded lancet 10 in FIG. 17 can now be grasped somewhat with a tweezer-like grip, would manipulate the lancet so that the points 14 and 22 is brought toward the skin area 15 containing the foreign object, as shown in FIG. 17, in the direction of arrow A. The user then applies sufficient pressure on the handles (in the direction of arrow D and E) such that point 14 or 22 then pierces the skin just under and over the foreign object 16. Since point 14 or 22 is a flat surface, when the gripped splinter is manipulated in an upward direction away from the skin (shown by the direction of arrow F) it can actually pull out the foreign object. A single point or pin cannot easily pull an object out because it has a single area and the object will tend to slide off the point.

In this regard, the lancet can also be manipulated to pierce the skin, adjacent and parallel to the splinter, in the direction of arrow A. The rear end of handle area 12 and 24 can then be pivoted downwardly away from the splinter (as shown by direction F), causing points 14 and 22 to move into or over and under the splinter. Removal of the lancet at this point makes it highly likely that the splinter can be dislodged.

Thus, while only one embodiment of the present invention has been shown and described, it is to be understood that many changes and modifications may be made thereunto without departing from the spirit and scope of the invention as defined in the appended

1. A method for removing a splinter from a patient's skin with a hand-held, disposable, elongated metal, generally flat lancet, having a handle portion and a point at both comprising:

2. The method of claim 1, wherein removing the lancet from an individually wrapped pre-sterilized package;

3. The method of claim 1, wherein piercing the patient's skin in an area adjacent to said splinter;

4. The method of claim 1, wherein tearing the patient's skin by piercing the skin adjacent to the splinter and then lifting the lancet in a direction perpendicular to the entry point in the patient's skin in a repetitive fashion so as to loosen the skin around the splinter;

5. The method of claim 1, wherein bending the lancet at bend line located at center between two points till the once handle are now tapered “V-shaped”, the free flat points being movable toward and from each other;

6. The method of claim 1, wherein placing said end engaging flat points of said lancet under and over said splinter;

7. The method of claim 1, wherein applying pressure to the now resilient based handles to grasp the splinter; and

8. The method of claim 1, wherein Thereafter, pulling it away from the skin so as to dislodge said splinter.

9. A disposable lancet comprised of a generally flat handle with a embossing along handle, wherein each end has a point, and a bending line located at center handle between the points:

10. The lancet of claim 9, wherein said lancet has a set of embossing along said handle portions to aid in manipulating the device.

11. The lancet of claim 9, wherein said lancet has a bending line located at center between two end tips to aid in bending and aligning the points.

12. The lancet of claim 9, wherein lancet is made from a thermoplastic material.

13. The lancet of claim 9, wherein lancet has a point one on either side.

14. The points of claim 13, wherein said points have flat angled edges.

15. The lancet of claim 9, wherein said points having slanted tips.

16. The tips of claim 15, wherein said slanted tips having an angle of about 45 degrees.

17. The lancet of claim 9, wherein said lancet handles are resilient based, at their said one end, applying pressure urges said points together.

18. The lance of claim 9, wherein said lancet handles are resilient based, at said one end, relieving applied pressure to handles urges said points apart.

19. The lancet of claim 9, wherein said lancet is made from a metal material.

20. The lancet of clam 9, wherein said lancet has a line located on handle at center between two points to aid in bending and aligning the edges.
